(19) United States
(12) Patent Application Publication
(10) Pub. No.: US 2022/0142562 A1
(43) Pub. Date: May 12, 2022

(54) CALCULATION AND ANALYSIS METHOD, PLANNING AND APPLICATION PLATFORM THAT PERSONALIZES THE MATHEMATICAL DEFINITION OF SPINAL ALIGNMENT AND SHAPE

(71) Applicants: Ahmet ALANAY, Istanbul (TR); ÌlyasÌ Çaglar YILGÖR, Istanbul (TR)

(72) Inventors: Ahmet ALANAY, Istanbul (TR); ÌlyasÌ Çaglar YILGÖR, Istanbul (TR)

(21) Appl. No.: 17/271,673

(22) PCT Filed: Dec. 31, 2018
(86) PCT No.: PCT/TR2018/000137

§ 371 Date: Feb. 26, 2021

(30) Foreign Application Priority Data
Aug. 28, 2018 (TR) 2018/12223

(51) Int. Cl. A61B 5/00; G16H 30/40; G16H 50/20; G16H 50/30

(52) U.S. Cl. CPC A61B 5/4566; G16H 50/30; G16H 50/20; G16H 30/40

(57) ABSTRACT

The present invention refers to a novel analysis method and application platform, utilized in fields of orthopedics and traumatology, neurosurgery, physiotherapy, and in related fields, that is used for preventive medicine applications and for the diagnosis and treatment of spinal disorders evaluating the standing spinal shape and alignment in a personalized manner based on the magnitude of the pelvic incidence of each individual, wherein the approach that utilizes population-based averages in calculation is abandoned.

### TECHNICAL FIELD

The present invention refers to a novel analysis method and application platform, utilized in the fields of orthopedics and traumatology, neurosurgery, physiotherapy, and in related fields, that is used for preventive medicine applications and for the diagnosis and treatment of spinal disorders, evaluating the standing spinal shape and alignment in a personalized manner based on the magnitude of the pelvic incidence of each individual, wherein the approach that utilizes population-based averages in calculation is abandoned.

### CURRENT STATUS

Spine is the weight-bearing skeleton of the body. While the spine in the ideal upright standing posture is straight in the frontal plane; it has various physiological curvatures in the sagittal plane such as cervical and lumbar lordosis, and thoracic kyphosis. Magnitude and location of these curvatures differ from one person to another. In order to have a good posture, to avoid spinal diseases, to improve the clinical results of spinal surgeries, and to prevent postoperative mechanical complications, preservation of the normal limits of these sagittal curvatures (sagittal alignment and shape) plays an essential role.

The prevalence of spinal disorders and degenerative diseases have increased due to rising number of desk jobs in parallel with recent technological advancements, emergence of postural disorders at younger ages in relation to changing practices of everyday life, and increase of average age within society. It is anticipated that spinal diseases will be of concern to 60% of people who are aged 60 years and above. When compared to cardiac diseases, diabetes, and chronic obstructive pulmonary disease (COPD), it was reported that spinal conditions affect the public health at least as much as these chronic diseases. Therefore, preservation of spinal health gains more and more importance for a person's quality of life.

Angulations of cervical, thoracic and lumbar curvatures are mathematically calculated for the planning of spinal surgeries and non-operative treatment applications. Measurements of sagittal plane curvatures and alignment in these anatomical regions are used, in general, for evaluation and surgical planning as well as physiotherapy and orthosis/prosthesis applications. Upper and lower ideal limits of these curvatures and alignments are calculated based on the population-based averages, and treatment objectives are determined to remain within these ranges.

Despite all said efforts of calculation and planning, dissatisfaction rates after non-operative treatment, and mechanical complication rates after spinal surgery are still high. When a surgical operation is planned according to the current, widely used population-based averages approach, mechanical complications can occur even when the obtained correction is within the recommended ranges. While the majority of complications resulting in mechanical problems comprises proximal junctional kyphosis and proximal junctional failures, complications such as distal junctional kyphosis and failure, adjacent segment degeneration, implant-related complications (screw loosening and breakage, hook, cage and screw pullouts, etc.), nonunion and rod fractures are also observed. Approximately fifty percent of patients who experience a mechanical complication requires a second intervention for a revision surgery. Despite repeat revision surgeries, ideal sagittal alignment still cannot be obtained in some patients.

Spinal fusion procedure is a commonly applied method in the surgical treatment of spinal diseases in which vertebrae are united together by stabilizing certain parts of the spinal column. Since the vertebral segment involved in the operated area for fusion will be stabilized in a certain position using rods, the position in which the stabilization will be carried out is important. Since the said position varies individually and according to the anatomy of the spinal segment involved in the surgical field, medical companies provide rods in a straight shape. These rods are contoured, by the surgeon, during the operation. Traditionally, the contour of the rod was decided through experience, sensation and by just looking at them, while current technological developments allow for the use of computer programs and mobile applications. These programs adopt approaches that utilize population-based averages (e.g. SRS-Schwab Classification and Roussouly's Sagittal Shape Classification) and provide surgical planning objectives accordingly. However, high mechanical complication and revision rates cannot be avoided despite obtaining the stabilization within the recommended target ranges.

Various studies indicate patient-related factors, technical factors and sagittal plane to be the most important factors affecting the development and prevention of mechanical complications. Most common errors in the sagittal plane, are the inability to achieve personalized cervical, thoracic and lumbar curvatures and shapes; and consequently, performing under- or overcorrection. This is due to surgical planning according to the current literature, established on the population-based averages. Trying to adapt an individual to the population average rather than respecting that individual's body measurements, and anatomical curvatures and shapes, results in over- or undercorrection instead of reaching an ideal correction, which leads up to mechanical complications.

The overall conclusion is that widely used current systems, which set targets for sagittal plane alignment using population-based averages, are inadequate. The criteria for the SRS-Schwab Classification are Pelvic Tilt (PT), Pelvic Incidence minus Lumbar Lordosis (PI−LL) and Sagittal Vertical Axis (SVA). For these three criteria, target values are the same for everyone and every patient's sagittal plane alignment correction are calculated in accordance with these targets. Roussouly classification, on the other hand, comprises five sagittal spine shapes that the corrections should be performed accordingly for a successful treatment. However, neither SRS-Schwab approach that considers all individuals as one type, nor Roussouly's approach which expresses that there are five different types, can provide guidance towards a treatment that perfectly matches to every individual's body posture and anatomical structure. Nevertheless, it is obvious that every individual has a unique anatomy. Thus, complications can be avoided by subject specific evaluation and personalized treatment approach.

The subject matter of invention, Global Alignment and Proportion Score (hereinafter referred to as “GAP”) that personalizes the calculation of radiographic spinal angular measurements for every individual's specific pelvic incidence using algorithmic mathematical formulas, reduces mechanical complication rates and prevents revision surgeries performed for such complications. Utilization of the GAP score for analysis and planning allows personalized planning in computer programs and mobile applications. Doing so, patient's quality of life improves and satisfaction increases. Since personalized treatment planning approach prevents complications and revision surgeries, it reduces the costs and provides further savings in terms of health planning and economics.

Research and Development activities, in this regard, are carried by many people and institutions. Previous patent applications to Turkish Patent and Trademark Office emphasize the importance of the studies performed in this field.

For example, in the patent registration request with the application number 2004/02686, and the title “Device For Fixing Bones, Particularly Vertebral Bodies, In Relation To One Another” “A device for fixing bones, particularly vertebral bodies in relation to one another comprising A) a longitudinal bearer (1), which has a central axis (2); and comprising B) n anchoring elements (3.i) (2<=I<=n) with longitudinal axes (4) and each having a front end (5) and a rear end (6), characterized in that G) at least the anchoring element (3.j) comprises a transport device (15) for inserting the anchoring element (3.j) into a bone parallel to the longitudinal axis (4).” is disclosed.

Another example is in the application filed with the number of 2017/10207 and under the title “Spine Stabilization Device” that discloses “A spine stabilization device is provided, the spine stabilization device comprising an interbody spacer (3) shaped to be inserted between a vertebral body (1) of an upper vertebra and a vertebral body (2) of a lower vertebra, and comprising a top surface (11) oriented towards the lower endplate of the vertebral body of the upper vertebra and a bottom surface oriented towards the upper endplate of the vertebral body of the lower vertebra; the interbody spacer comprising at least one channel-like recess (123) reaching to an end in the top surface and at least one channel-like recess (123) reaching to an end in the bottom surface, and comprising in a region of these recesses a structure (124) that includes an undercut, whereby it is suitable for making a positive-fit connection together with an anchoring device (121). The spine stabilization device further comprises for every channel-like recess an anchoring device (121), the anchoring devices comprising a proximal end and a distal end, a first securing portion (127), a second securing portion (127) and a bridge portion (128) between the first and second securing portions.”

### CURRENT IMPLEMENTATIONS IN THE TECHNICAL FIELD

In non-surgical spinal treatments and spine surgery, it is crucial to maintain and restore normal sagittal alignment for improving clinical outcomes and preventing mechanical complications. Currently, SRS-Schwab classification sagittal modifiers (PT, PI−LL, SVA) are widely used in order to restore sagittal spinal alignment. These criteria use angular radiographic measurements as absolute values and categorize them as normal, moderate and severe. Treatment goals are set to have the patients classified as ‘normal’ for all three modifiers. The magnitude of required changes in different spinal segments can be calculated in one's head, or various computer programs can be used through uploading radiographs to the digital media.

Today, various applications such as Surgimap, KEOPS, SpineEOS, X Align and BACS have been developed for measuring radiographs on digital media and simulating surgeries. Surgimap, which has versions for Windows, MAC, Cloud, Android and iOS, is the oldest and the most widely used program among others. Since KEOPS is integrated into a data storage system, which requires an annual subscription for measurements and simulations, it has not achieved extensive usage. KEOPS works as a web-based system and does not have mobile or computer applications. SpineEOS works only with an imaging system called EOS imaging, while X Align operates only with a navigation device called Mazor X, and both systems are available in very few centers around the world. In a relatively new system called BACS or Balance ACS, which has not yet been introduced to daily use, there are modules for surgical planning, data collection and 3D-printing preparation. Mutual disadvantages of all the abovementioned systems are that they use either SRS-Schwab Classification or Roussouly Types for calculations and determination of target criteria, which adopt population-based approaches. Due to said reasons in Current Status section, the use of population-based averages approach is not adequate in preventing mechanical complications.

When the SRS-Schwab Classification is used as target values in preoperative evaluation of radiographs and surgical planning, mechanical complication and revision rates cannot be reduced despite the use of computer-assisted technological applications. The main reason for this is that the targeted criteria for the alignment are determined according to the HRQoL (Health-Related Quality of Life Questionnaire) results rather than biomechanical and anatomical characteristics. These target values have been determined by considering population-based averages and are the same for all individuals. The descriptors of these scientific rules reported, in their own articles, that one out of every three patients have experienced a mechanical complication, and half of those who experienced a mechanical complication had to undergo reoperation.

Roussouly Types, which is another sagittal plane definition method, in contrast to Schwab Classification, is not based on numerical measurements, but on visual descriptions. It describes the shapes of the curvatures by defining the apex of the curvature and the inflection point between opposing curvatures. It also delineates the number of spinal segments within each curvature. In this approach, five different spine types with distinguishable characteristics have been defined, in normative databases. The KEOPS system can be used to monitor whether the patient complies with one of these predefined spine types or not and to plan treatments. However, this application, apart from the accessibility issue, faces two other difficulties. First one, is the difficulty of determining the original spine type, once age-related degeneration occurs. Second one, is the difficulties experienced in terms of interpretation and explication, since this is an analysis method based on visuals. Although the correlation between Roussouly spine types and mechanical complications has not been demonstrated yet, it may be considered that it will not prevent mechanical complications since it utilizes population-based averages.

### BRIEF DESCRIPTION OF THE FIGURES

FIG. 1 illustrates a representation of the Personalized Analysis, Planning and Application Platform.

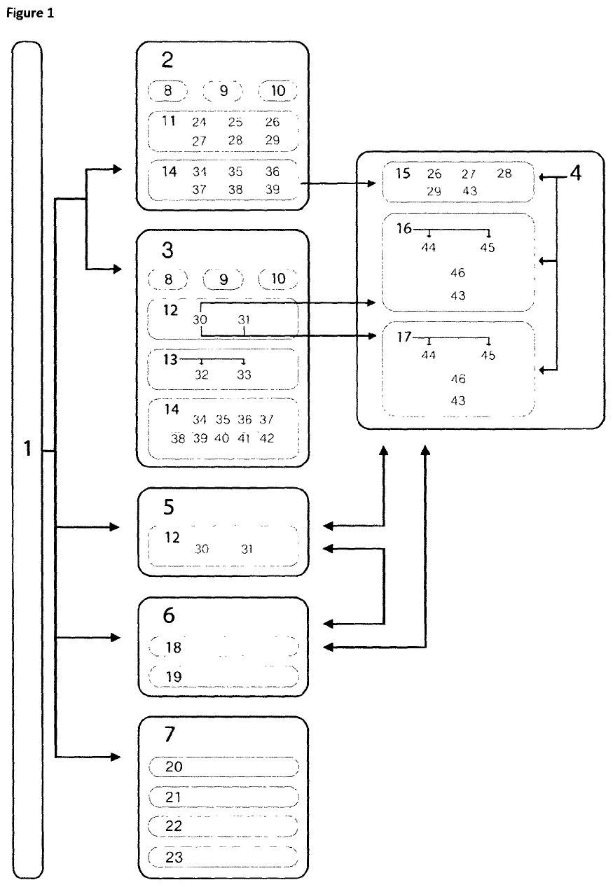

FIG. 2 is an illustration of the spinal regions and GAP alignment as described in the present application.

FIG. 3 is an illustration of RPV as described in the present application.

FIG. 4 is an illustration of RLL as described in the present application.

FIG. 5 is an illustration of LDI as described in the present application.

FIG. 6 is an illustration of RSA as described in the present application.

FIG. 7 is an illustration of GAP as described in the present application.

### DETAILED DESCRIPTION OF THE INVENTION

Reference numbers in Figure:

1—Access Module

2—GAP Score Calculation Module

3—GAP Radiograph Analysis Module

4—Personalized Treatment Planning Module

5—Treatment Evaluation Module

6—Data Storage Module

7—Adaptation Module

8—Patient Data

9—Medical Record Number

10—Date

11—Measurement Input Field

12—Radiograph Upload Interface

13—Radiograph Marking Interface

14—Results Field

15—Delta Planning

16—Two-Dimensional Planning

17—Three-Dimensional Planning

18—Comparison Module

19—Printing Module

20—Study Design Module

21—Data Collection Module

22—Radiograph Matching Module

23—Statistical Analysis Module

24—Age

25—Pelvic Incidence

26—Sacral Slope

27—L1-S1 Lordosis

28—L4-S1 Lordosis

29—Global Tilt

30—Lateral Radiograph

31—Anterior-Posterior Radiograph

32≥Artificial Intelligence Function

33—Manual Function

34—Age Factor

35—Relative Pelvic Version

36—Relative Lumbar Lordosis

37—Lordosis Distribution Index

38—Relative Spinopelvic Alignment

39—GAP Score

40—T2-T12 Kyphosis

41—T5-T12 Kyphosis

42—T10-L2 Angle

43—Risk Assessment Module

44—Manual Mode

45—Guidance Mode

46—Pre-Bend Rod Module

The present invention relates to a novel calculation and analysis method, planning and application platform that personalizes the evaluation of the standing sagittal spinal alignment for every magnitude of the pelvic incidence. The present invention abandons the currently used population-based averages approach and adopts a personalized medicine approach in the field of spine health and diseases.

The fact that even the creators of the currently used classification systems, which determine the correction criteria for the sagittal plane, report high mechanical complication and revision rates, revealed the necessity of a novel approach for the interpretation of the sagittal plane. Especially the studies carried out by French researchers revealed the fact that angles measured in sagittal plane affect each other in a chain of correlations. The concept of chain of correlations implies that the amount of every spinal curvature affects the amount of the next curvature. According to this concept, a person's lumbar lordosis is affected by the sacral kyphosis; thoracic kyphosis is affected by lumbar lordosis; and cervical lordosis is affected by the thoracic kyphosis. Pelvis is considered to be the foundation of the spine, interconnecting the spine to the legs. Sagittal angular width and tilt of the pelvis is in close relation with the abovementioned sagittal plane spinal curvatures. For example, a patient with a larger horizontal diameter of the pelvis will have a more tilted pelvis while standing; a patient with a more tilted pelvis will have a deeper lumbar lordosis; a patient with a deeper lordosis will have a larger thoracic kyphosis; and a patient with a larger kyphosis will have a deeper cervical lordosis, and vice versa.

All these relations and chain of correlations described are based on data collected from asymptomatic individuals who reported no spinal problems. Once bones, joints, discs, ligaments and other soft tissues lose their anatomical and physiological qualities via accidents, various spinal diseases, and through normal aging process, associations and correlations between the pelvis and the spine and among spinal curvatures begin to deteriorate. Majority of spinal diseases as well as normal aging, cause physical deformation and impairments that lead to postural disturbances, forcing a person to stand in a forwardly leaned manner. However, human beings, through subconscious reflex mechanisms, have tendencies to stand in the upright posture, to position their heads over the pelvis, and to maintain a forward gaze. This is the most ergonomic position that leads to least energy consumption while standing and walking.

In cases where spine and the upright posture deviate from this ergonomic “ideal”, so-called compensatory mechanisms are activated, that subconsciously use reserves, in order to maintain upright posture and forward gaze. Lexical meaning of the word “compensation” is a process in which the change in a given direction is counteracted by another conscious or unconscious change. Reserves used to compensate spinal malalignment, are located in the spine and in non-spinal body segments (particularly legs). Use of these reserves requires active muscle contraction by the subject and increases energy consumption related to standing and walking.

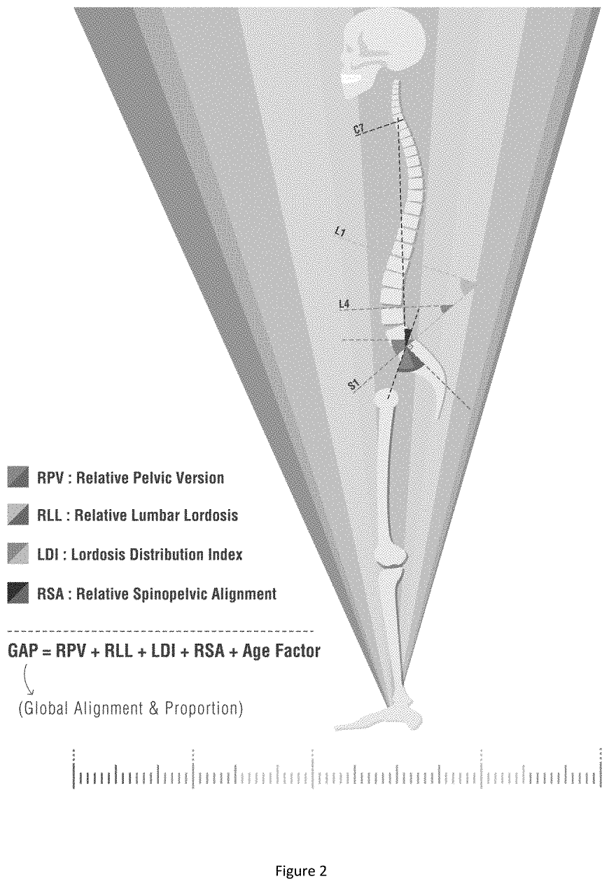

The subject matter of invention, novel sagittal plane calculation and analysis method, of which the pilot study was carried out in the European Spine Study Group (ESSG) database, is based on the ground of the fact that pelvis is the foundation of the spine and physiological sagittal plane curvatures are shaped according to the pelvis. This method is named as Global Alignment and Proportion (GAP) score, as shown in FIG. 2. This method reveals that in cases of degenerated spine, where the compensations are in use, absolute numeric values of all measured angles in radiographs can be misleading, except for one of these angles.

Among all other measured angles, the only angle that can remain constant during the adult life without being affected by diseases and aging is the Pelvic Incidence (PI). This is because of the pelvis' completely osseous structure that contains no joints with significant motion, and because it is not affected by soft tissues. This angle represents the angular width between the center of femoral heads and the upper end-plate of the sacrum. It is a mathematical measure of the horizontal diameter of the pelvis. Normative data studies reveal that the Pelvic Incidence (PI) angle varies between 20 and 90 degrees.

In the concept of GAP analysis, since the Pelvic Incidence does not change during the adult life, it is considered as a signature of a given individual. Since all other angles measured from radiographs are affected by diseases and age-associated degeneration, their absolute values cannot be directly used. In calculations and analyses, which are the subject of this invention, all sagittal plane parameters are subject to assessment in proportion to PI and calculated as deviations from “ideal” in a personalized manner. As such, angular measurements of the physiological cervical, thoracic and lumbar curvatures are not considered as absolute values, but as relative personalized values. For example, let's assume 3 different patients whose lumbar lordosis angles are measured as 50°, wherein the ideal lumbar lordosis calculated according to their pelvic anatomy are 40°, 50° and 60°. Although all three patients have the same lumbar lordosis angle, because their ideals are different, lumbar lordosis angles of these three patients are actually not the same. Relatively, one patient lacks 10 degrees of lordosis, while the other one has an excess of 10 degrees. Personalized interpretation of lumbar lordosis simplifies the quantification as deviations from the calculated ideal. In addition to defining angles as relative values, the concept involves defining distribution and shape to facilitate visual perception. On the other hand, when the same three patients are subjected to evaluation with the PI−LL parameter within the scope of population-based averages, 50 degrees of lumbar lordosis is classified as “normal”. This complicates the interpretation of the patient's deformity.

The concept of using angles as relative angular values by personalizing them for every individual's specific Pelvic Incidence value via algorithmic mathematical formulas, instead of using measured absolute values, forms the basis of the GAP analysis. Personalized GAP analysis correlates the mechanical complications to the numerical value of the GAP score that indicates the deviation from ideal and the amount of compensation used. As such, the GAP analysis system defines, for the first time, the tolerable amount of compensation for each fused spinal segment. As the amount of deviation from the ideal exceeds tolerable limits, an imbalance between biological and mechanical factors affecting the healing process arises, leading up to mechanical complications. Contrary to approaches of SRS-Schwab which utilizes HRQoL results and Roussouly that uses normative data, personalized GAP approach is developed in a spinal deformity database, which involves patients treated for spinal disorders, by taking biomechanical properties and mechanical complications into consideration.

GAP analysis clearly distinguishes whether the identified presence and magnitude of deviations from ideal are resulted from diseases, pathology and deformity or from compensation; thus, allows personalized and more accurate decision-making in surgical planning.

Although SRS-Schwab and Roussouly approaches are directly or indirectly utilized for the planning of spinal treatments and avoiding of mechanical complications, both of these approaches were not actually developed for this particular purpose. GAP analysis and planning system, wherein relative angular values denote deviations from the calculated ideal, is the first system developed for prediction of mechanical complications and for prevention of these complications by preoperative planning.

The second fundamental part of the subject matter of invention is a personalized treatment planning and application platform. This platform allows for calculating angles measured from spine radiographs as relative deviations from the ideal, and planning personalized treatments, and controlling planned treatments both during and after the treatment. Hence, it is possible to determine via interim evaluations whether the targeted values are achieved, and if not, to perform interventions during the course of the treatment or surgery. Post-treatment or post-operative evaluations within this platform, by performing a risk assessment, can also determine whether the targets are reached or not, allowing to perform early interventions and taking protective measures in order to prevent complications, when the ideal values have not been reached.

This application platform which operates on web, computer and mobile devices, depicts algorithmically calculated compensation-free ‘true’ deformity and algorithmically calculated ideal, and compares these with the patient's current condition, by using algorithmic formulated Relative Pelvic Version, Relative Lumbar Lordosis, Lordosis Distribution Index and Relative Spinopelvic Alignment parameters that constitute the GAP score. These comparisons provide an opportunity to make a distinction between deformities and compensations developed in response. This distinction is of utmost importance for the success of the treatment. Thus, it facilitates the visual identification of problematic segments on the sagittal plane of the spine in addition to providing numeric data. This application facilitates treatment planning via making the plan and correction suggestions on compensation-free “true deformity”; not on compensated deformities. Formerly, since deformities and compensations cannot be clearly distinguished in approaches that utilize population-based averages, another precaution taken for the prevention of complications was including more and more spinal segments into the operated area. The GAP approach, on the other hand, suggests operating the deformities and not the compensations. A patient whose deformities are corrected will not be in need of any compensation, and consequently, these compensations will resolve. Therefore, the GAP analysis allows obtaining better results by involving fewer spinal segments in the operated area.

### ADVANTAGES OF THE INVENTION

Main advantage of the present invention is that it develops personalized interpretation of sagittal shape and alignment instead of using population-based averages. Calculations and analyses of all sagittal plane parameters except for Pelvic Incidence (PI), which are the subject of this invention, are assessed in proportion to PI, and calculated as deviations from the “ideal” in a personalized manner. As such, angular measurements of the physiological cervical, thoracic and lumbar curvatures are not considered as absolute values, but as relative personalized values. Personalized GAP analysis correlates the mechanical complications to the numerical value of the GAP score that indicates the deviation from ideal and the amount of compensation used. These comparisons provide an opportunity to make a distinction, which is crucial for the success of the treatment, between deformities and compensations, allowing to identify the main reason behind the spinal impairment more accurately, by facilitating the perception of the spinal malalignment. Personalized GAP approach is developed in a spinal deformity database, which involves patients treated for spinal disorders, by taking biomechanical properties and mechanical complications into consideration. As such, the GAP analysis system defines, for the first time, the tolerable amount of compensation for each fused spinal segment.

Another advantage of the present invention is that the subject matter method is the first and only method that includes personalized evaluation of all pelvic, lower and total arc lordosis, and global alignment in a single score, for any given individual.

The most important practical advantage of the present invention is that the GAP score, which personalizes angular spinal radiographic measurements by algorithmic mathematical formulations, reduces the rate of postoperative mechanical complications and the necessity of recurrent spinal surgeries performed in association with these complications. Because it provides a personalized surgical planning, not only it reduces the rate of mechanical complications, but it also acts as a time-buying strategy for the development of these complications. GAP analysis and planning system, wherein relative angular values denote deviations from the calculated ideal, is the first system developed for prediction of mechanical complications and for prevention of these complications by personalized preoperative planning. In the pilot study that involves 222 patients (168 Female, 54 Male) registered in the European Spine Study Group database who underwent at least four-level spinal fusion and had at least 2 years of follow-up, the GAP score calculated from postoperative early radiographs was able to predict 92% of the mechanical complications developed after a mean follow-up of 29 months. It demonstrated the fact that the mechanical complication rates reported as being thirty percent when surgery planning is performed according to population-based averages can be reduced down to six percent through personalized planning.

Another advantage of the present invention is that personalized treatment planning calculates the use of compensatory mechanisms and allows taking preventive measures by predicting postoperative high-risk groups. Doing so, treatment options are better assessed while planning spinal surgeries and post-treatment quality of life can be improved. The application, which is the subject matter of invention, is the first system that assists decision-making by performing risk assessment for the potential complications and allows taking necessary precautions by predicting high-risk groups before the risk actualizes.

Personalized sagittal plane analysis, planning and application platform automatically calculates the GAP score, its parameters, ideal values and required changes in treatment planning, and offers these in one single application. By utilization of the GAP score, the subject matter of invention is the first simulation program that allows personalized planning. Another advantage of the invention is that it is an application, operating in computers and mobile devices, which algorithmically calculates the personalized GAP score in the background from simple angular values measured manually or automatically by using artificial intelligence, and visualizes the spinal alignment in accordance with the calculated values, and allows simulating the treatment using these visuals.

Another advantage of the subject matter of invention is that, in addition to being able to handle vertebrae one by one for a detailed planning, it allows harmonic and successive planning of predefined anatomic spinal segments (Sacrum, lower arc lordosis, upper arc lordosis, thoracolumbar junction, lower arc kyphosis, upper arc kyphosis, cervicothoracic junction and cervical lordosis) as a whole.

Another advantage of the present invention is that the application platform which operates on web, computer and mobile devices, depicts algorithmically calculated compensation-free ‘true’ deformity and algorithmically calculated ideal, and allows to compare these with the patient's current condition, by using algorithmically formulated Relative Pelvic Version, Relative Lumbar Lordosis, Lordosis Distribution Index and Relative Spinopelvic Alignment parameters that constitute the GAP score.

Other advantages of the subject matter of the invention's calculation, planning and application platform are; providing automatic calculation of the GAP score and the GAP parameters, depicting deviations of the GAP parameters from ideals, evaluating the sagittal plane radiographs in digital environment, enabling to perform shorter surgical operation by means of operating deformity and not the compensation, simulating personalized surgical treatment that yields high success rate, calculating the GAP score of the result of the simulated surgery, predicting estimated mechanical complication rates of the simulated surgery, and indicating achievement of the target values by comparing intraoperative radiographs and simulation.

### DISCLOSURE OF THE INVENTION

The subject matter of invention comprises two main sections: 1) An algorithmic calculation and analysis method; 2) A planning and application platform. The novelty of the planning and application platform is that it utilizes algorithmic calculation and analysis method. The GAP score, first part of the invention, is a method that performs pelvic incidence-based personalized analysis instead of population-based averages. Second part of the invention is a web, computer and mobile application that calculates the algorithmically formulated parameters, which constitute the GAP score via simple angular radiographic measurements, facilitates the sagittal plane analysis, and allows treatment planning in digital environment.

### Definition, Calculation and Updatable/Modifiable Structure of the GAP Score

The Global Alignment and Proportion (GAP) score is a pelvic incidence-based proportional score that assesses the sagittal shape and alignment. The GAP method is the first and only method that evaluates the spinal column altogether by including all pelvic, lower and total arc lordosis and global alignment elements into a single score, that is tailored for each individual.

The GAP score calculates the deviation of the measured radiographic angular values from the calculated personalized ideals. Ideal Sacral Slope is calculated by PI×0.59+9 formula; Ideal Lumbar Lordosis by PI×0.62+29 formula, and Ideal Global Tilt by PI×0.48−15 formula.

These formulas which are utilized in the calculation of the parameters constituting the GAP score have an updatable and modifiable structure. For example, abovementioned formulas have been calculated from the “Washington University in St. Louis” asymptomatic volunteers database through logistic regression analysis. Numerous ideal values for various populations and diseases can be defined through information acquired from multiple normative databases that include different races and populations such as Japanese, Africans, etc. whose anatomical features and daily life activities are different. Similarly, for various age groups such as children, teenagers, etc. who has different anatomical and physiological characteristics, different ideal values can be defined.

PI-based proportional GAP score comprises Relative Pelvic Version (RPV=Measured−Ideal Sacral Slope), Relative Lumbar Lordosis (RLL=Measured−Ideal Lumbar Lordosis), Lordosis Distribution Index (LDI=L4-S1 Lordosis/L1-S1 Lordosis×100), Relative Spinopelvic Alignment (RSA=Measured−Ideal Global Tilt) and age factor. Each radiographic parameter is divided into either aligned or 3 subgroups of disproportioned that displayed maximum intergroup and minimum intragroup heterogeneity with regards to mechanic complications, where Chi-squared values reached maximum within the same degree of freedom.

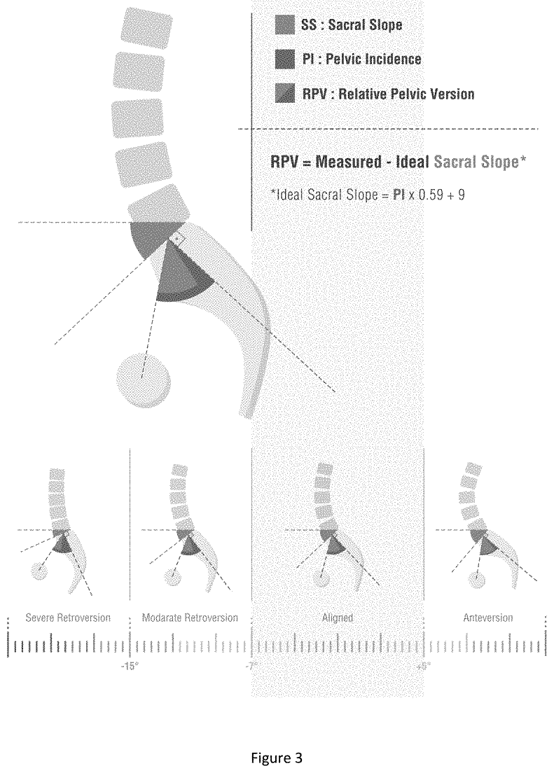

As shown in FIG. 3, relative pelvic version indicates the spatial orientation of the pelvis relative to the ideal sacral slope as defined by the magnitude of PI. RPV<−15° was considered severe retroversion, −15°≤RPV<−7°as moderate retroversion, −7°≤RPV≤5° as aligned and RPV>5° as anteversion.

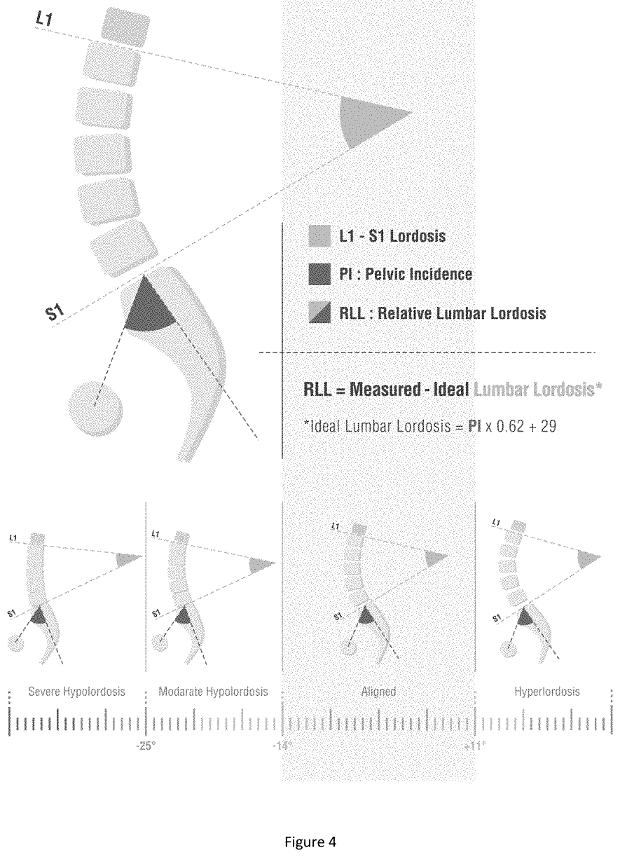

Relative lumbar lordosis indicates the amount of lordosis relative to the ideal lordosis as defined by the magnitude of PI as shown in As shown in FIG. 4. RLL<−25° was considered severe hypolordosis, −24°≤RLL<−14° as moderate hypolordosis, −14°≤RLL≤11° as aligned and RLL>11° as hyperlordosis.

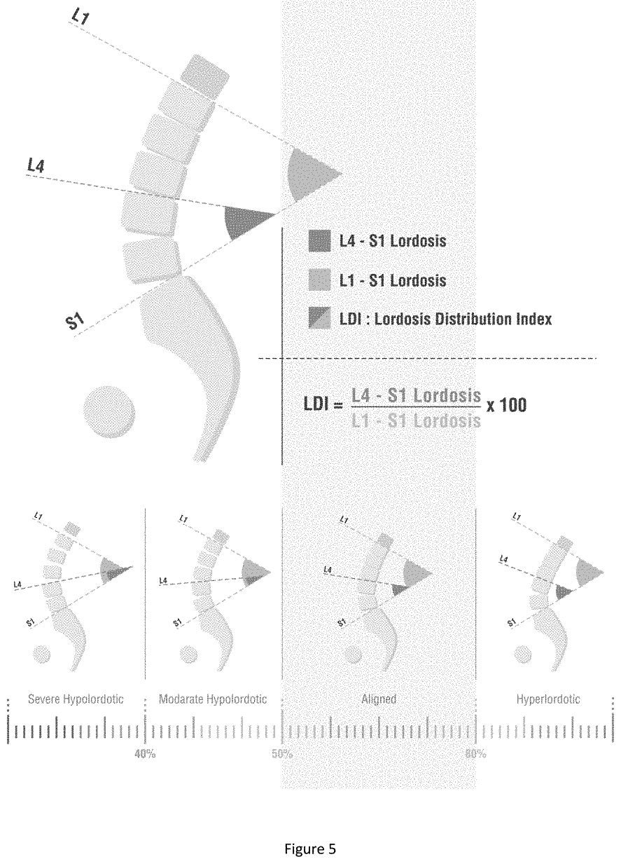

As shown in FIG. 5, the lordosis distribution index defines the amount of lower arc lordosis in proportion to total lordosis. LDI≤40% was considered severe hypolordotic maldistribution, 40%≤LDI≤49% as moderate hypolordotic maldistribution, 50%≤LDI≤80% as aligned and LDI>80% as hyperlordotic maldistribution.

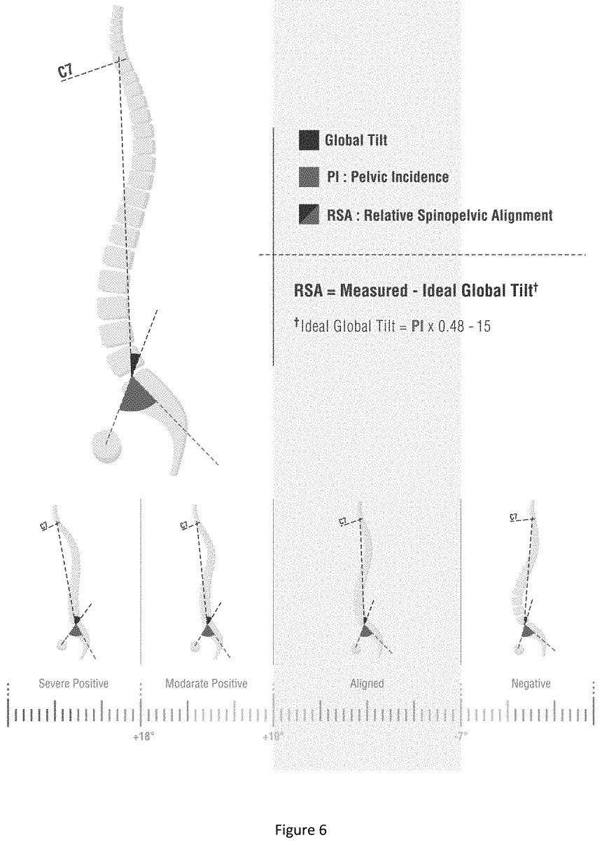

As shown in FIG. 6, relative spinopelvic alignment indicates the amount of malalignment relative to the ideal global tilt as defined by the magnitude of PI. RSA>18° was considered severe positive malalignment, 10°≤RSA<18° as moderate positive malalignment −7°≤RSA≤10° as aligned and RSA<−7° as negative malalignment.

Similar to the calculation of the ideal values, there is also an updateable and modifiable structure in the calculation of the parameter cutoff points. For instance, abovementioned cutoff points have been calculated by using European Spine Study Group database. Number of patients registered to this database increases day by day. Within this modifiable/updateable structure, cutoff points will be defined more accurately with decreased margins of error as the number of registered patients to the database increase. Using various databases comprising pre- and postoperative follow up data, different cutoff points can be defined for different populations, age groups and diseases.

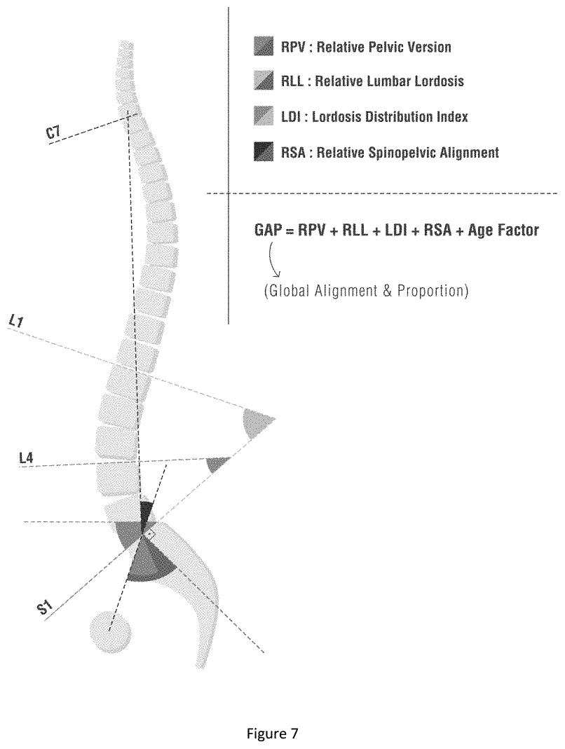

Odds ratios for mechanical complication are statistically calculated for parameter subgroups, which are defined according to the amount of positive and negative deviation from the ideal. Logarithms (β regression factor) of these odds ratio are calculated, and the result is rounded to the nearest integer in order to determine the score of each subgroup. Scores of the radiographic parameters vary between 0 and 3. Score of the age factor varies between 0 and 1. As shown in FIG. 7, the GAP score is calculated by adding the scores obtained from the radiographic parameters and age factor. GAP score varies between 0 and 13. A GAP score of 0-2 was categorized as proportioned (GAP-P), 3-6 as moderately disproportioned (GAP-MD) and ≥7 as severely disproportioned (GAP-SD).

Similar to ideals and cutoff points, determination of scores for the GAP parameter subgroups and categorization of the GAP score are also updateable and modifiable. ROC (Receiver Operating Characteristic) curve of the GAP score versus mechanical complications is used for categorization of the score. When various ideal values and cutoff points defined for different populations, age groups and diseases are used, values in each category of proportioned, moderately and severely disproportioned also differ.

By using this methodology in modifying the ideal values used in the calculation of the GAP parameters, cutoff points and categories, the GAP score is universally used in a personalized manner. These updates are performed through proven scientific statistical methods. The basis of the score is created via abovementioned statistical methods such as logistic regression, chi-squared, odds ratio, β regression factor and ROC curve. There are various additional methods that are used for “fine tuning” and broadening the scope of the score. Kaplan-Meier and COX regression analyses evaluate the effect of the duration of follow-up. Apart from the biostatistical methods described above, scope of the score is further broadened by using medical informatics methods. Methods utilized herein are generally known as artificial intelligence or machine learning applications. “Machine Learning” which evaluates the interactions by direct examination of the data without biases and clinical information, and “Deep Learning” that empowers its own prediction abilities via self-training as new data is added, are big data analysis methods used in the universal implementation of the GAP analysis. GAP analysis method maintains its ever changing and up-to-date structure by means of updating cutoff points and formulations of the GAP score in accordance to changing population, surgical methods and materials through the use of biostatistics and bioinformatics.

### DETAILED DESCRIPTION

### Details of Personalized Analysis, Planning and Application Platform

Personalized analysis, planning and application platform allows personalized treatment planning through the analysis of sagittal radiographs in digital environment for spinal physiotherapy, brace and surgery. The functionalities of this web, computer and mobile apps vary depending on the platform used. Thus, the presence and absence of modules detailed below differ for different platforms.

Personalized sagittal plane analysis, planning and application platform automatically calculates the GAP score, its parameters, ideal values and required changes in treatment planning, and offers these in one single application. It is an application that runs in computers and mobile devices that algorithmically calculates the personalized GAP score in the background from simple angular values measured manually or automatically by using artificial intelligence, and visualizes the spinal alignment in accordance with the calculated values, and allows simulating the treatment using these visuals. Simulating surgeries according to the GAP concept, the application denotes potential mechanical complication risks before the surgery is performed, helping prevent such complications. By utilizing the GAP score, this application platform, which is the subject matter of invention, is the first simulation program that performs personalized surgical planning.

Upon launching the application platform, the Access Module (1) is viewed. This module comprises username and password fields and various related features. After singing in, radiographic measurements are entered in the GAP Score Calculation Module (2); or radiographs are uploaded to the GAP Radiograph Analysis Module (3).

The GAP Score Calculation Module (2) has an interface consisting of input fields such as Patient Data (8), Medical Record Number (9) and Date (10). Once the abovementioned data input is complete, the Measurement Input Field (11) is accessed. Then, measurement values such as Age (24), Pelvic Incidence (25), Sacral Slope (26), L1-S1 Lordosis (27), L4-S1 Lordosis (28) and Global Tilt (29) are entered. The Results Field (14) displays automatically calculated results for Age Factor (34), Relative Pelvic Version (35), Relative Lumbar Lordosis (36), Lordosis Distribution Index (37), Relative Spinopelvic Alignment (38) and GAP Score (39), which are algorithmically formulated in a personalized manner for every individual's specific pelvic incidence. Calculated Values (a), Scales (b) and Attributed Scores (c) for GAP parameters (module numbers 34-38), and Calculated Values (a) and Category (b) for GAP score (module number 39) are provided. Values and scores are presented as numeric data, while scale demonstrates the GAP parameters' deviation from the ideal on a colored legend chart.

The GAP Radiograph Analysis Module (3) has an interface consisting of input fields such as Patient Data (8), Medical Record Number (9) and Date (10). Once the abovementioned data input is complete, the Radiograph Upload Interface (12) is accessed. Lateral Radiographs (30) alone, or together with Anterior-Posterior Radiographs (31) can be uploaded using this interface. Once radiographs are uploaded, the Radiograph Marking Interface (13) is accessed. Radiographs can be marked automatically using the Artificial Intelligence Function (32), or manually using the Manual Function (33). The Artificial Intelligence Function (32) automatically detects femoral heads, sacrum upper end plate, C7, L1 and L4, and the spatial locations of these bony landmarks, and their interrelation. This function, using deep learning algorithms, improves accuracy as new radiographs are uploaded, decreasing the margin of error. Once automatic or manual marking is completed, the Results Field (14) displays automatically calculated results for Age Factor (34), Relative Pelvic Version (35), Relative Lumbar Lordosis (36), Lordosis Distribution Index (37), Relative Spinopelvic Alignment (38) and GAP Score (39), which are algorithmically formulated. Calculated Values (a), Scales (b) and Attributed Scores (c) for GAP parameters (module numbers 34-38), and Calculated Values (a) and Category (b) for GAP score (module number 39) are provided. In addition, T2-T12 Kyphosis (40), T5-T12 Kyphosis (41) and T10-L2 angle (42) are provided.

After the results are viewed, the Personalized Treatment Planning Module (4) is accessed. This module comprises Delta Planning (15), Two-Dimensional Planning (16) and Three-Dimensional Planning (17) interfaces.
In case that the calculations are performed in the GAP Score Calculation Module (2), the treatment planning is carried out on the Delta Planning Interface (15). This interface shows numeric amounts of deviations from the ideals, and required minimum and maximum corrections for Sacral Slope (26), L1-S1 Lordosis (27), L4-S1 Lordosis (28) and Global Tilt (29). When a correction that is out of recommended correction range is planned, the magnitude of the desired correction amount can be entered into the relevant fields. Then, the Risk Assessment Module (43) is accessed, in which potential risk for mechanical complications and confidence intervals are displayed, should the surgery be performed with the manually entered correction amounts. Various surgical options can be simulated, entering different values, until an acceptable risk for the user is reached.
In case that the calculations are performed in the GAP Radiograph Analysis Module (3) uploading only Lateral Radiographs (30) to the Radiograph Upload Interface (12), the treatment planning is carried out on the Two-Dimensional Planning Interface (16). This interface illustrates a two-dimensional spine model in the sagittal view. Angulations for each spinal segment can be altered using this model, as well as performing displacement in antero-posterior and supero-inferior directions. Cages and various surgical instruments, and surgical techniques such as chevron, pedicle subtraction osteotomy and vertebral column resection are predefined on the interface. Surgeries are simulated in the Manual Mode (44) by entering surgical instruments and planned correction steps. The orientation of each vertebra can be altered angularly or in antero-posterior and supero-inferior planes. Postoperative spinal alignment and the risk of mechanical complications of the simulated surgery in the Manual Mode (44), are displayed using GAP parameters and GAP score data. Various surgical options can be simulated, entering different surgical plans, until the anticipated GAP score and/or an acceptable risk of complications, for the user, are reached. Guided surgical simulation is performed in the Guidance Mode (45), using values and scales of the GAP parameters. Simulation starts by correcting the spatial orientation of the pelvis. Then, L4-S1 and L1-S1 Lordosis are sequentially set to the ideal values. Finally, global alignment is corrected via manipulating the thoracic kyphosis and cervical lordosis taking into account the personalized anatomical structure of the patient. After the completion of the step-by-step guided simulation, considering the differences between the current and the ideal, the user chooses amongst various surgical instruments and techniques for different anatomical regions. For a more detailed planning, vertebrae can be handled one by one, or predefined anatomic spinal segments (Sacrum, lower arc lordosis, upper arc lordosis, thoracolumbar junction, lower arc kyphosis, upper arc kyphosis, cervicothoracic junction and cervical lordosis) can be simulated in an harmonic and successive way as a whole. After the completion of the planning in the Manual Mode (44) and the Guidance Mode (45), rod bending is visualized in accordance with selection of fusion levels. The Pre-Bend Rod Module (46), allows two or three-dimensional real-scale printing of the rods to match the curvatures for the simulated correction. At any given point during the simulation using either the Manual Mode (44) or the Guidance Mode (45), the Risk Assessment Module (43) can be accessed, in which potential risk for mechanical complications and confidence intervals are displayed, should the surgery be performed in accordance with the current stage of the simulation. Various surgical options can be simulated, entering different values, until an acceptable risk for the user is reached.
In case that the calculations are performed in the GAP Radiograph Analysis Module (3) uploading both Lateral Radiographs (30) and Anterior-Posterior Radiographs (31) to the Radiograph Upload Interface (12), the treatment planning is carried out on the Three-Dimensional Planning Interface (17). This interface illustrates a three-dimensional spine model that can be rotated around any given gauge point. In addition to all available features in the Two-Dimensional Planning Interface (16) described above, this interface allows planning corrections also in the anterior-posterior plane. As the user can rotate the view at any given point, the interpretation of the current and the ideal spinal shape and alignment is facilitated.

By uploading the intraoperative radiographs to the Treatment Evaluation Module (5), treatment being performed is compared with the simulated plan in the Personalized Treatment Planning Module (4). Mismatch between the planned and the performed treatment is automatically calculated using the same interface previously used for planning [Delta (15), Two-Dimensional (16) or Three-Dimensional (17)]. All abovementioned functions are used to calculate the required modifications. Decisions can then, be made using the Risk Assessment Module (43) in the relevant interface of the Personalized Treatment Planning Module (4). Thus, the user is allowed to make modifications and adjustments before finalizing the surgery or the treatment.

Another module of the planning and application platform is the Data Storage Module (6). The previously recorded data and planning details can be accessed through this module. Reports can be generated in the Comparison Module (18) and the Printing Module (19). Comparisons can be made between the preoperative status and the simulated or performed treatments as well as amongst various treatment options. Likewise, radiographs obtained in different time points during the follow-up of a patient can also be compared. Flexibility can be evaluated by comparing standing and side-lying sagittal radiographs, using personalized analysis parameters of the relevant anatomic spinal regions. Changes observed between standing to side-lying radiographs are calculated automatically and provided as absolute and percentage values for the parameters provided in modules numbered from 34 to 42. This information assists the selection of various surgical instruments and techniques for different anatomical regions while performing treatment planning in the Manual Mode (44) and the Guidance Mode (45). Thus, a more realistic, applicable and accessible treatment can be planned. Details of a single analysis and simulation, or results of comparative analyses performed in the Comparison Module (18) can be printed in the Printing Module (19). Printouts include all numeric data and scales given the Measurement Input Field (11) and the Results Field (14) as well as visual outputs. Preferably, results obtained in the Risk Assessment Module (43) can also be included in the printouts.

Final module of the planning and application platform is the Adaptation Module (7). This module creates data-specific personalized GAP score calculations for site-specific patient profiles and surgical preferences. Using various criteria such as age, diagnosis, surgery type, clinical and radiographic data, the Study Design Module (20) determines the inclusion and exclusion criteria to create a personalized GAP score. Indicating data to be used among demographical data, comorbidities, background information, surgical details and mechanical complications, a data collection interface is created in the Data Collection Module (21). It is compulsory to add some data types, while others are subject to preference. The Radiograph Matching Module (22) automatically or manually matches radiographs recorded in the database with the patient data. Radiographs are classified as preoperative, early postoperative and follow-up. The Statistical Analysis Module (23) primarily measures the performance of the currently available GAP score in the relevant data by using the collected data and measurements obtained from the matched radiographs. Chi-Squared is used to compare continuous data, while Kruskal Wallis is used for categorical data, Cochran-Armitage for the determination of complication trends, multivariate logistic regression tests for the determination of risk ratios, and area under the curve, specificity, sensitivity, positive and negative predictive values and accuracy in classification for diagnostic performance measures. Subsequently, if deemed necessary, a data-specific GAP score can be created through specifying cutoff points for deviation from ideals, and scores specific to this data by using the methodology described in the Calculation of the GAP Score section. This module only runs if predefined minimum number of patients and minimum duration of follow-up is achieved.

1. An application platform that can be used for preventive medicine applications and for the diagnosis and treatment of spinal disorders and further be utilized in the fields of orthopedics and traumatology, neurosurgery, physiotherapy and in related fields, wherein the approach that utilizes population-based averages in the calculation is abandoned, and instead, standing spinal shape and alignment is evaluated in a personalized manner, based on the magnitude of the pelvic incidence of every individual; comprising:
a Global Alignment and Proportion (GAP) Score Calculation Module that performs personalized evaluation of all sagittal plane components together, and includes pelvic, lower and total arc lordosis, and global alignment in a single score, for any given individual.

2. The platform according to claim 1, further comprising at least one GAP Radiograph Analysis Module that automatically detects and measures angular radiographic values.

3. The platform according to claim 2, further comprising at least one Artificial Intelligence Function within a Radiograph Marking Interface that uses deep learning algorithms to automatically detect and mark bony landmarks.

4. The platform according to claim 3, further comprising at least one Personalized Treatment Planning Module that includes at least one Delta Planning, at least one Two-Dimensional Planning, and at least one Three-Dimensional Planning interfaces.

5. The platform according to claim 4, further comprising at least one Results Field where the results for Age Factor, Relative Pelvic Version, Relative Lumbar Lordosis, Lordosis Distribution Index, Relative Spinopelvic Alignment and GAP Score are shown via a personalized calculation of radiographic spinal angular measurements for every individual's specific pelvic incidence using algorithmic mathematical formulas.

6. The platform according to claim 5, further comprising at least one scale which demonstrates the GAP parameters' deviation from the ideal on a colored legend chart.

7. The platform according to claim 6, further comprising the process steps of:
calculating of updateable ideal formulas through the linear logistic regression from the normative databases for the personalized description of spatial orientation of the pelvis, magnitude and distribution of lumbar lordosis and global spinal alignment; evaluating all sagittal plane parameters in proportion to pelvic incidence by taking into account the relation between radiographic angular measurements and calculated ideals, and calculating P1-based radiographic parameters that defines radiographic measurements as personalized deviations from the ideal; determining of the cutoff points of parameter subgroups by Chi-Squared analysis; determining odds ratios for mechanical complication within these subgroups; calculating the logarithm of these odds ratios; determining the scores of each subgroup by rounding the logarithmic values to the nearest integer; calculating GAP score by adding these scores and determining GAP categories by calculating the area under the ROC curve.

8. The platform according to claim 7, further comprising Relative Pelvic Version (RPV) angle, defined as the subtraction of Ideal Sacral Slope from the Measured Sacral Slope in radiographs, for the evaluation of the spatial orientation of the pelvis within P1-based proportional GAP concept.

9. The platform according claim 9, further comprising Relative Lumbar Lordosis (RLL) angle, defined as subtraction of Ideal Lumbar Lordosis from the Measured Lumbar Lordosis in radiographs, for the evaluation of the magnitude of lumbar lordosis within P1-based proportional GAP concept.

10. The platform according to claim 9, further comprising Lordosis Distribution Index (LDI), defined as the division of L4-S1 Lordosis to the L1-S1 Lordosis multiplied by 100, for the evaluation of the distribution of lumbar lordosis within P1-based proportional GAP concept.

11. The platform according to claim 10, further comprising Relative Spinopelvic Alignment (RSA) angle, defined as the subtraction of the Ideal Global Tilt from the Measured Global Tilt in radiographs, for the evaluation of global spinopelvic alignment within P1-based proportional GAP concept.

12. The platform according to claim 11, further comprising at least one Guidance Mode in which guided treatment planning can be performed using values and scales of the GAP parameters.

13. The platform according to claim 12, further comprising at least one Risk Assessment Module which can, at any given point during the simulation using either the Manual Mode or the Guidance Mode, display potential risk for mechanical complications and confidence intervals, should the surgery be performed in accordance with the current stage of the simulation.

14. The platform according to claim 13, further comprising at least one Comparison Module that allows the evaluation of flexibility, various treatment options, treatment results, and changes during the follow-up by performing comparisons between standing and side-lying sagittal radiographs, preoperative status and the simulated or performed treatments or various treatment options, and radiographs obtained in different time points during the follow-up, respectively.

15. The platform according to claim 14, wherein the platform is capable of being updatable and modifiable to meet the changing needs of various populations, surgery methods and surgical materials through the use of biostatistics and bioinformatics, for ideals used in the calculation of GAP parameters, scores attributed to parameter subgroups, and score ranges used in the categorization of the GAP score.

16. The platform according to claim 15, further comprising at least one Adaptation Module that creates data-specific personalized GAP score calculations for site-specific patient profiles and surgical preferences.
